(19) United States
(12) Patent Application Publication
(10) Pub. No.: US 2007/0238732 A1
(43) Pub. Date: Oct. 11, 2007

(54) BRIMONIDINE AND TIMOLOL COMPOSITIONS

(75) Inventors: Richard Graham, Irvine, CA (US); Rhett M. Schiffman, Laguna Beach, CA (US); Brent A. Johnson, Ladera Ranch, CA (US); Patrick M. Hughes, Aliso Viejo, CA (US)

Correspondence Address: ALLERGAN, INC., 2525 DUPONT DRIVE, T2-7H, IRVINE, CA, 92612-1599, US

(73) Assignee: Allergan, Inc.

(21) Appl. No.: 11/681,150

(22) Filed: Mar. 1, 2007

Related U.S. Application Data
(60) Provisional application No. 60/744,550, filed on Apr. 10, 2006.

(51) Int. Cl. A61K 31/5377; A61K 31/4164

(52) U.S. Cl. 514/235.5; 514/396

(57) ABSTRACT

Disclosed herein are compositions comprising brimonidine and timolol. Methods and medicaments related thereto are also disclosed.

### CROSS-REFERENCE TO RELATED APPLICATION

This application is based on, and claims priority under 35 U.S.C. § 120 to U.S. Provisional Patent Application No. 60/744,550 filed on Apr. 10, 2006, and which is incorporated herein by reference.

### DESCRIPTION OF THE INVENTION

Disclosed herein is a composition comprising brimonidine having a concentration from about 1 mM to about 4.5 mM and timolol having a concentration from about 2 mM to about 15.8 mM, wherein the pH of said composition is from 7 to about 8.5.

Also disclosed herein is a composition comprising brimonidine having a concentration from about 1 mM to about 4.5 mM and timolol having a concentration from about 2 mM to about 16 mM, wherein the pH of said composition is from 7 to about 8.5.

This composition is useful for treating glaucoma or reducing elevated intraocular pressure.

Also disclosed herein is a medicament for the treatment of glaucoma or ocular hypertension by topical administration to an eye of a mammal, said medicament comprising brimonidine having a concentration from about 1 mM to about 4.5 mM and timolol having a concentration from about 2 mM to about 15.8 mM, wherein the pH of said medicament is from 7 to about 8.5.

Also disclosed herein is a method comprising topically administering a composition to an eye of a mammal, said method being useful for the treatment of glaucoma or ocular hypertension, wherein said composition comprises brimonidine having a concentration from about 1 mM to about 4.5 mM and timolol having a concentration from about 2 mM to about 15.8 mM, wherein the pH of said composition is from 7 to about 8.5.

Also disclosed herein is use of a composition in the manufacture of a medicament for the treatment of glaucoma or ocular hypertension, said composition comprising brimonidine having a concentration from about 1 mM to about 4.5 mM and timolol having a concentration from about 2 mM to about 15.8 mM, wherein the pH of said composition is from 7 to about 8.5.

Also disclosed herein is a kit containing a composition, a container for dispensing drops of said composition, and instructions for administration of said composition topically to an eye of a person, wherein said composition comprises brimonidine having a concentration from about 1 mM to about 4.5 mM and timolol having a concentration from about 2 mM to about 15.8 mM, wherein the pH of said composition is from 7 to about 8.5.

Brimonidine is a compound having the formula shown below. It is commercially available from Allergan, Inc. for the treatment of glaucoma or ocular hypertension in the form of the tartrate salt as Alphagan® (0.2% brimonidine tartrate) or Alphagan P® (0.15% brimonidine tartrate). However, for the purposes of this disclosure, “brimonidine” refers to any salt of brimonidine, not just the tartrate, as well as the free base.

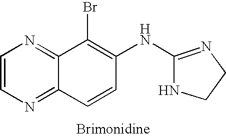

One composition comprises brimonidine tartrate.

The concentration 4.5 M brimonidine tartrate is lower than 0.2% brimonidine tartrate.

In another composition the concentration of brimonidine is from about 2 mM to about 3.5 mM.

In another composition the concentration of brimonidine is about 3.4 mM.

In another composition the concentration of brimonidine is about 2.26 mM.

Timolol is a compound having the formula shown below. It is commercially available from a number of sources, generally in the form of a solution at a concentration of 0.5% or 0.25% of the maleate salt. However, for the purposes of this disclosure, “timolol” refers to any salt of timolol, not just the maleate, as well as the free base.

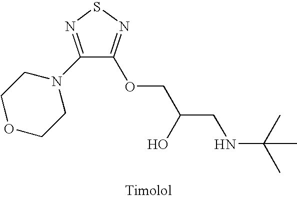

In one composition, the concentration of timolol is about 15.8 mM or about 7.9 mM.

In another composition, the concentration of timolol is from about 7 mM to about 15.8 mM.

The abbreviation “mM” refers to millimolar concentration, i.e. 10⁻³ M, as generally recognized in the art. For a liquid which is not a homogenous liquid, such as an emulsion, the millimolar concentration is taken to include the number of millimoles of the compound divided by the volume of the liquid in liters. The volume of the liquid is the volume of the entire liquid, including all oil and water phases.

While not intending to be limited by theory, we have discovered that a composition comprising 0.5% timolol maleate and 0.2% brimonidine tartrate is not completely stable at a pH of 6.9. Over a period of months, one or more newly formed impurities have been discovered in these compositions when they are stored and/or transported under normal conditions. These impurities may become problematic such that the useful shelf life of these compositions may be significantly reduced. While not intending to be bound in any way by theory, it is believed that compositions having timolol maleate and brimonidine may be significantly more stable at a pH of 7 or greater. It is also believed that in the pH range of 7 to 8.5 small increases in pH may have substantial effects upon the stability of the compositions. Thus, the presently claimed compounds are substantially more useful because they are expected to improve the shelf life of the compositions.

In one composition the pH is from about 7.4 to about 8.5.

In another composition the pH is from about 7.8 to about 8.5.

In another embodiment, the composition is not a composition containing about 0.2% brimonidine tartrate by weight and about 0.5% timolol by weight.

A liquid which is ophthalmically acceptable is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions are often maintained at a comfortable pH with an appropriate buffer system. The formulations may also contain conventional, pharmaceutically acceptable preservatives, stabilizers and surfactants.

Various buffers and means for adjusting pH may be used so long as the resulting preparation is ophthalmically acceptable. Accordingly, buffers include, but are not limited to, acetate buffers, citrate buffers, phosphate buffers and borate buffers. Acids or bases may be used to adjust the pH of these formulations as needed.

Preservatives that may be used in the pharmaceutical compositions disclosed herein include, but are not limited to, benzalkonium chloride, chlorobutanol, thimerosal, phenylmercuric acetate and phenylmercuric nitrate.

Certain compositions contain solubility enhancing components (SECs) in amounts effective to enhance the solubility of brimonidine at a given pH. These SECs may be anionic in nature, and can be polymeric in nature. In one embodiment the SEC is a cellulose derivative, in another embodiment the SEC is not a cellulose derivative or a cyclodextrin. In these compositions, the SEC is used to enhance the solubility of brimonidine. In other words, in two compositions containing brimonidine which are identical except for the presence of an effective amount of the SEC, more brimonidine will be dissolved in the composition containing the SEC than the in the composition not containing the SEC.

The SEC may include a non-ionic or polyanionic component. As used herein, the term “polyanionic component” refers to a chemical entity, for example, an ionically charged species, such as an ionically charged polymeric material, which includes multiple discrete anionic charges. Non-ionic SECs may include polyvinyl alcohol (PVA), polyvinyl pyrrolidone (povidone), and various gums and other non-ionic agents.

In one embodiment, the SEC is a polyanionic component, which may be selected from polymeric materials having multiple anionic charges, and mixtures thereof.

Examples of useful polyanionic components are selected from anionic polymers derived from acrylic acid (meaning to include polymers from acrylic acid, acrylates and the like and mixtures thereof), anionic polymers derived from methacrylic acid (meaning to include polymers from methacrylic acid, methacrylates, and the like and mixtures thereof), anionic polymers derived from alginic acid (meaning to include alginic acid, alginates, and the like and mixtures thereof), anionic polymers of amino acids (meaning to include polymers of amino acids, amino acid salts, and the like and mixtures thereof), and the like, and mixtures thereof. Very useful polyanionic components are those selected from anionic cellulose derivatives and mixtures thereof, especially carboxymethyl cellulose and its derivatives.

A surfactant may be used for assisting in dissolving an excipient or an active agent, dispersing a solid or liquid in a composition, enhancing wetting, modifying drop size, or a number of other purposes. Useful surfactants, include, but are not limited to sorbitan esters, Polysorbate 20, Polysorbate 40, Polysorbate 60, Polysorbate 80, stearates, glyceryl stearate, isopropyl stearate, polyoxyl stearate, propylene glycol stearate, sucrose stearate, polyethylene glycol, polyethylene oxide, polypropylene oxide, polyethylene oxide-polypropylene oxide copolymers, alcohol ethoxylates, alkylphenol ethoxylates, alkyl glycosides, alkyl polyglycosides, fatty alcohols, phospholipids, phosphatidyl chloline, phosphatidyl serine, and the like.

Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components which may be included in the ophthalmic preparations are chelating agents. A useful chelating agent is edetate disodium, although other chelating agents may also be used in place or in conjunction with it.

Compositions may be aqueous solutions or emulsions, or some other acceptable liquid form. For an emulsion, one or more oils will be used to form the emulsion, and in some instances one or more surfactants and/or emulsion stabilization excipients will be required. Suitable oils include, but are not limited to anise oil, castor oil, clove oil, cassia oil, cinnamon oil, almond oil, corn oil, arachis oil, cottonseed oil, safflower oil, maize oil, linseed oil, rapeseed oil, soybean oil, olive oil, caraway oil, rosemary oil, peanut oil, peppermint oil, sunflower oil, eucalyptus oil, sesame oil, and the like.

In one embodiment, the composition has no α,β-unsaturated carboxylic acid or salt thereof. An α,β-unsaturated carboxylic acid is a carboxylic acid which wherein the carboxylcarbon is directly attached to a doubly or triply bonded carbon, e.g., —C(H)═C(H)—CO₂H, —C≡C—CO₂H, a salt thereof, or the like. In another composition, no maleic acid or maleate salt is present.

In another composition, no carboxylic acid or salt thereof is present.

### EXAMPLE 1

### Aqueous solution compositions may be prepared according to Table 1.

TABLE 1

| | Formulation | | | | | |
|---|---|---|---|---|---|---|
| | 1 | 2 | 3 | 4 | 5 | 6 |
| Brimonidine Tartrate (mM) | 3.39 | 3.39 | 2.26 | 2.26 | 2 | 2 |
| Timolol Maleate, EP (mM) | 15.8 | 15.8 | 15.8 | 7.90 | 7.90 | 7.90 |
| Benzalkonium Chloride, NF, | 50 | 50 | 50 | 50 | 50 | 50 |
| EP (ppm) | | | | | | |
| Sodium Phosphate, monobasic | 0.43 | 0.43 | 0.43 | 0.43 | 0.43 | 0.43 |
| monohydrate, USP (% w/v) | | | | | | |
| Sodium Phosphate, dibasic | 2.15 | 2.15 | 2.15 | 2.15 | 2.15 | 2.15 |
| heptahydrate, USP (% w/v) | | | | | | |
| Sodium Hydroxide, NF | 7.0 | 7.4 | 7.8 | 8 | 8.2 | 8.5 |
| (adjust to pH shown) | | | | | | |
| Hydrochloric Acid, NF (adjust | 7.0 | 7.4 | 7.8 | 8 | 8.2 | 8.5 |
| to pH shown) | | | | | | |
| Carboxymethylcellulose | — | 0.5 | 1 | 1.5 | 2 | 2 |
| (% w/v) | | | | | | |
| Purified Water, USP, EP | q.s. | q.s. | q.s. | q.s. | q.s. | q.s. |
| | ad | ad | ad | ad | ad | ad |

### EXAMPLE 2

Emulsions are formulated with the compositions shown in Table 2 using the method described in U.S. Pat. No. 5,981,607, incorporated herein by reference, with the brimonidine and timolol being added to the castor oil before introducing the oil into the emulsion.

| | Formulation | | |
|---|---|---|---|
| | 1 | 2 | 3 |
| Brimonidine Tartrate (mM) | 3.39 | 2.26 | 2.26 |
| Timolol Maleate, EP (mM) | 15.8 | 15.8 | 7.90 |
| Castor Oil (% w/w) | 1.25 | 1.25 | 1.25 |
| Polysorbate 80 (% w/w) | 1.0 | 1.0 | 1.0 |
| Pemulen ® (% w/w) | 0.1 | 0.1 | 0.1 |
| Glycerin (% w/w) | 1.0 | 1.0 | 1.0 |
| Boric Acid (% w/w) | 0.6 | 0.6 | 0.6 |
| Purite ® (% w/w) | 0.0075 | 0.0075 | 0.0075 |
| Purified Water | qs.ad. 100 | qs.ad. 100 | qs.ad. 100 |

### EXAMPLE 3

A patient suffering from elevated intraocular pressure or glaucoma is treated with a composition of Example 1 or 2. The composition is administered topically to the eyes of the patient twice a day. Within a few hours reduction in pressure is observed, and an acceptable pressure is achieved within one or two days. Normal intraocular pressure is maintained for as long as the patient receives the composition twice a day.

What is claimed is:

1. A composition comprising brimonidine having a concentration from about 1 mM to about 4.5 mM and timolol having a concentration from about 2 mM to about 16 mM, wherein the pH of said composition is from 7 to about 8.5.

2. The composition of claim 1 wherein the concentration of brimonidine is from about 2 mM to about 3.5 mM.

3. The composition of claim 1 wherein the concentration of timolol is about 15.8 mM or about 7.9 mM.

4. The composition of claim 2 wherein the concentration of brimonidine is about 3.4 mM.

5. The composition of claim 2 wherein the concentration of brimonidine is about 2.26 mM.

6. The composition of claim 1 wherein the pH is from about 7.4 to about 8.5.

7. The composition of claim 5 which further comprises a solubility enhancing component.

8. The composition of claim 1 wherein no maleic acid or maleate salt is present.

9. The composition of claim 7 wherein no carboxylic acid or salt thereof is present.

10. The composition of claim 1, wherein the pH is from about 7.8 to about 8.5.

11. The composition of claim 1 which is an emulsion.

12. A medicament for the treatment of glaucoma or ocular hypertension by topical administration to an eye of a mammal, said medicament comprising a composition according to any one of claims 1 to 12.

13. A method comprising topically administering a composition according to any one of claims 1 to 12 to an eye of a mammal in need thereof, said method being useful for the treatment of glaucoma or ocular hypertension.

14. Use of a composition according to any one of claims 1 to 12 in the manufacture of a medicament for the treatment of glaucoma or ocular hypertension.
